(19) United States
(12) Patent Application Publication
(10) Pub. No.: US 2021/0128001 A1
(43) Pub. Date: May 6, 2021

(54) EKG MONITORING DEVICE FOR USE WITH SMART WATCH

(71) Applicant: Fresnel Technologies Inc., Fort Worth, TX (US)

(72) Inventor: Richard N. Claytor, McKinney, TX (US)

(21) Appl. No.: 17/089,529

(22) Filed: Nov. 4, 2020

Related U.S. Application Data
(60) Provisional application No. 62/931,309, filed on Nov. 6, 2019.

(51) Int. Cl. A61B 5/0402; A61B 5/00

(52) U.S. Cl. CPC A61B 5/0402; A61B 5/743; A61B 5/681; A61B 5/0006

(57) ABSTRACT

A system is provided for taking an electrocardiogram of a subject. The system includes a wireless communications system; a first device which is disposable on a first limb of the subject, said first device containing a first tangible, non-transient memory device and a first sensor which measures a first electrophysiological signal of the user; a second device which is disposable on a second limb of the subject, said second device containing a second sensor which measures a second electrophysiological signal of the user and which is in communication with said first device via said wireless communications system; and a software program which is resident in said first memory device and which produces an electrocardiogram from the first and second electrophysiological signals measured by the first and second sensors.

### CROSS-REFERENCE TO RELATED APPLICATION

This application claims the benefit of U.S. provisional application No. 62/931,309, filed Nov. 6, 2019, having the same title, and having the same inventors, and which is incorporated herein by reference in its entirety.

### FIELD OF THE DISCLOSURE

The present disclosure pertains generally to EKG monitoring devices, and more particularly to systems and methodologies for pairing such a device with a smart watch to provide a multi-electrode EKG monitoring system.

### BACKGROUND OF THE DISCLOSURE

Many individuals have an electrocardiogram (EKG) (also referred to as an ECG) taken on a regular or ongoing basis. This includes, for example, patients suffering from cardiac arrhythmia or from other medical issues that affect the heart, as well as athletes and other individuals who wish to monitor their cardio fitness.

Various devices have been developed to date which are intended to allow users to obtain an EKG without the assistance of a medical professional. For example, the Qardiocore wearable ECG/EKG device allows users to capture EKG data, share this data with a healthcare provider, and track the data on the user's iPhone. Similarly, the Apple Watch Series 4 is equipped with an EKG function which has been approved by the FDA. It generates a PDF of the user's heart rhythm that can be shared with a medical professional or caregiver.

Various external heart monitors have also been developed, some of which may be paired to a smart watch. For example, the Wahoo Tickr™ is an external heart rate monitor which may be paired directly with the Apple iWatch to track heart rate or sync heart rate data through the Wahoo 7 Minute Workout app or Wahoo RunFit™ app.

### SUMMARY OF THE INVENTION

In one aspect, a system is provided for taking an electrocardiogram of a subject. The system comprises (a) a wireless communications system; (b) a first device which is disposable on a first limb of the subject, said first device containing a first tangible, non-transient memory device and a first sensor which measures a first electrophysiological signal of the user; (c) a second device which is disposable on a second limb of the subject, said second device containing a second sensor which measures a second electrophysiological signal of the user and which is in communication with said first device via said wireless communications system; and (d) a software program which is resident in said first memory device and which produces an electrocardiogram from the first and second electrophysiological signals measured by the first and second sensors.

In another aspect, a method is provided for monitoring the cardio state of a subject. The method comprises (a) disposing a first device on a first limb of the subject, wherein said first device is a mobile communications device equipped with a first sensor for capturing EKG data from the subject; (b) disposing a second device on a second limb of the subject, wherein said second device is equipped with a second sensor for capturing EKG data from the subject, and wherein said second device is in wireless communications with said first device; (c) receiving captured EKG data from the first and second sensors; (d) operating on the received data to generate at least one graphical object illustrating the cardio state of the subject; and (e) displaying the graphical object on a display associated with the first device. In some embodiments, the method may further comprise (f) sharing the received data with one or more healthcare professionals.

### BRIEF DESCRIPTION OF THE DRAWINGS

FIG. 1 is an illustration of an embodiment of an EKG monitoring system in accordance with the teachings herein shown disposed on the limbs of a user.

FIG. 2 is an illustration of the software architecture of the system of FIG. 1.

### DETAILED DESCRIPTION

While existing external heart rate monitors may be suitable for certain purposes, at present, many of these devices do not provide true or accurate EKG functionality. In order to provide accurate EKG data, it is typically necessary for a device to read data from two or more electrodes disposed on different parts of a subject's body. However, many external heart rate monitors are in the form of a singular band which is worn, for example, on the user's arm. Even if these devices are equipped with multiple electrodes, the proximity of the electrodes to each other limits the accuracy of the resulting EKG data.

Some EKG monitoring devices have been developed in the art which utilize two or more electrodes disposed on disparate parts of the user's body. For example, one type of such a device is a vest which contains multiple EKG electrodes. The vest ensures proper placement at different locations of the user's body. A similar effect is achieved by the aforementioned Qardiocore wearable ECG/EKG device. However, both of these devices are objectionable to many users because their presence is noticeable to the user and to others. Moreover, devices of this type are uncomfortable to wear over prolonged periods of time.

There is thus a need in the art for an EKG monitoring device which can provide accurate and reliable EKG data. There is further a need in the art for such a device which is comfortable to wear and which is unobtrusive to the user during use. These and other needs may be met with the systems, devices and methodologies disclosed herein.

In a preferred embodiment, the systems disclosed herein comprise a first device which is equipped with a first EKG electrode and which is disposed on a first part of the user's body, and a second device which is equipped with a second EKG electrode and which is disposed on a second part of the user's body. In an especially preferred embodiment, the first device is an external heart monitor, and the second device is a smart watch which is in communication with (and which is preferably paired or synced with) the external heart monitor. Preferably, the external heart monitor and the smart watch interact to function as a single EKG device which receives EKG signals from the first and second electrodes and which processes those signals in real-time or near real-time. Readouts of the processed data may be depicted in suitable graphical form on the user's smartwatch, smart phone or other synced device, and appropriate alerts may be issued to the user, to healthcare providers, or to other suitable parties of interest.

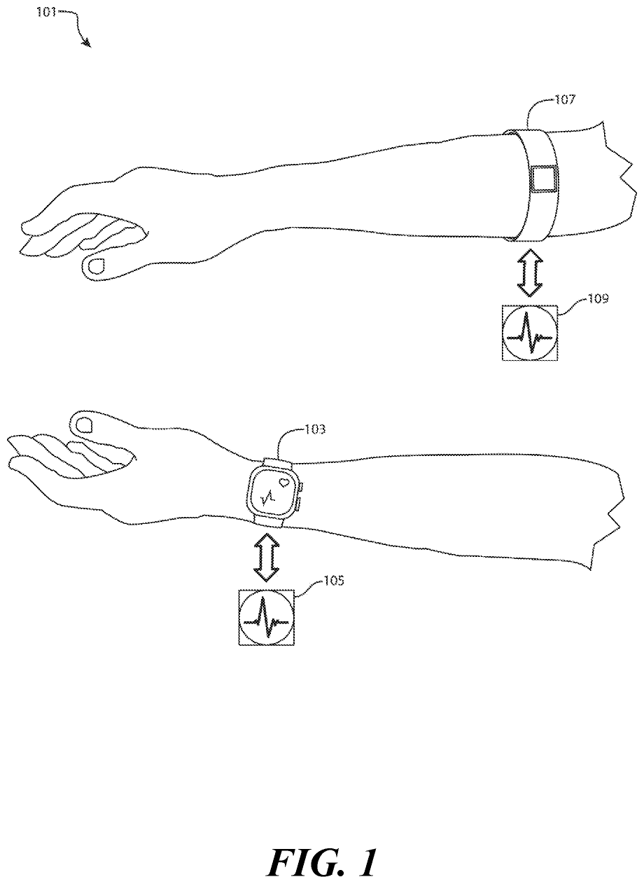

FIG. 1 shows a particular, non-limiting embodiment of a heart monitoring system in accordance with the teachings herein. The particular system 101 depicted therein comprises a smart watch 103 equipped with an EKG electrode 105. The system 101 further comprises an external heart monitor 107 which is equipped with a second EKG electrode 109. The external heart rate monitor 107 is in wireless communication with the smart watch 103, preferably via a suitable wireless communication protocol such as Bluetooth or RF.

The smartwatch has a software program (preferably in the form of a software client) resident thereon which includes suitable programming to handle communications with the external heart monitor and to collect and display EKG data received from the first 105 and second 109 EKG electrodes.

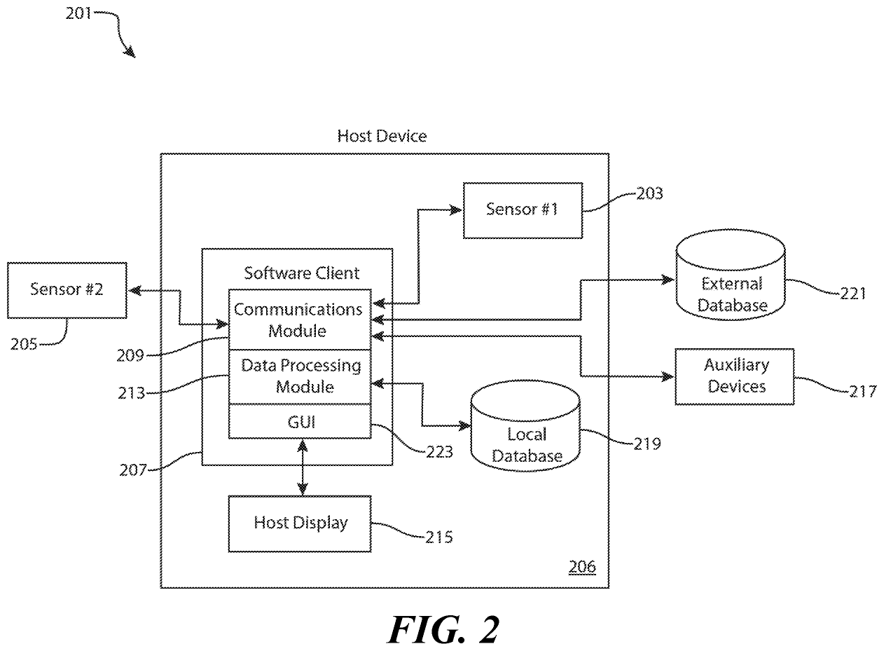

The functionality of the software client may be appreciated with respect to FIG. 2, which depicts a particular, non-limiting embodiment of a system in accordance with the teachings herein. As seen therein, the system 201 in the particular embodiment depicted comprises first 203 and second 205 sensors which are in communication with a device 206 having a software client 207 installed thereon. The first sensor 203 in the particular embodiment depicted is native to the device 206 (which is preferably a smartwatch), and the second sensor 205 is external to the device 206 (in the particular embodiment depicted, the second sensor 205 is incorporated into an external heart monitor paired with the device 206). The software client 207 includes a communications module 209, a data processing module 211 and a graphical user interface (GUI) 213.

The communications module 209 implements all necessary communications protocols and processes to allow the software client 207 to obtain data collected by, or generated from, the first 203 and second 205 sensors. These may include, for example, the implementation of suitable wireless communications protocols (such as, for example, Bluetooth or WiFi protocols) and the associated communications processes.

The data processing module 211 accepts data passed to it by the communications module 209 and operates on the data as appropriate to generate EKG signals. Such operations may include, for example, the application of noise filters to remove external noise from the data, the boosting of signal-to-noise ratios, the implementation of statistical functions, and the like. The refined data is then passed to the GUI 213 for display.

The GUI 223 accepts data from the data processing module 211, operates on the data as necessary to render graphical data therefrom, and ports the graphical data to the host display 215 or to auxiliary devices 217 for rendering. Such graphical data may include the data necessary for rendering objects such as graphs or charts depicting or summarizing the refined EKG data or aspects thereof. The GUI may include various menus or software objects to allow a user to interact with the software client 207. These menus or software objects may allow a user to specify what data is displayed on the host display 215, control the manner in which the data is displayed, or perform other suitable functions as are commonly performed by GUIs.

The software client 207 may write data to, or retrieve data from, one or more local databases 219 or external databases 221. For example, the software client 207 may write refined data to a local database resident on the device 206 as that data is processed. The software client 207 may then back up the data to an external database 221 periodically, at the request of the user, or whenever the communications module 209 has an established link to the external database 221 via a communications network. In some embodiments, the external database 221 may be maintained by, or accessible to, a healthcare provider associated with the user.

The systems, methodologies and devices disclosed herein may be utilized to take an ECG at any time such as, for example, under the control of a timer, in response to external stimuli, or in response to the user's physiological state. For example, in some embodiments, the systems, methodologies and devices disclosed herein may be configured to alert the user, and/or healthcare providers or personnel associated with the user, whenever irregular pulse patterns are detected, and such events may trigger the initiation of an EKG (if one is not already underway). Such irregular pulse patterns may include, but are not limited to, sinus rhythms, atrial fibrillation (AFib), heart rates that are below or above a threshold value, or readings that are inconclusive. In some embodiments, the alert may be accompanied by a relevant medical explanation and advice.

In preferred embodiments, the systems, methodologies and devices disclosed herein are configured to continuously record, and then (unless otherwise directed) erase, a short duration of an EKG. Consequently, if an alert of the type described above is generated, an EKG may be captured that includes readings from the time preceding the even that triggered the alert, as well as readings captured during and/or after the event. This information may allow a user or healthcare provider to understand the event in context and to draw better conclusions about its implications with respect to the user's physiological state.

Various modifications are possible to the systems, devices and methodologies disclosed herein without departing from the scope of the present disclosure. For example, while these systems, devices and methodologies disclosed herein have been principally illustrated with respect to a two-electrode configurations, one skilled in the art will appreciate that these systems, devices and methodologies may be readily modified to accommodate the use of more than two electrodes. Similarly, while these systems, devices and methodologies have been illustrated with a particular placement on the body of a first and second device containing EKG electrodes, one skilled in the art will appreciate that these devices may be placed in various positions on the body of the user without departing from the scope of the present disclosure. In some embodiments, for example, one or more devices containing an EKG electrode may be placed at various locations on the user's body through the use of various fasteners including, for example, hook-and-loop type fasteners, or through the use of bio-acceptable repositionable adhesives. Preferably, these devices are placed on the limbs of the users.

In some embodiments, groups of devices containing EKG electrodes may be utilized to implement the systems and methodologies described herein. These groups of devices may include, for example, smart watches and external heart rate monitors. For example, in some embodiments of this type, the group of devices may include a smart watch and multiple external heart rate monitors. Each of these devices may include a tangible, non-transient memory and a software client, an instance of which is recorded in the memory of each of the plurality of devices. The software client in such embodiments preferably contains suitable programming instructions which, when implemented by at least one processor of at least one computational device, implements a hyper distribution communications protocol among the devices and maintains a decentralized and distributed database of transactions between the devices. This results in a mesh network which can tolerate network disruptions and implement suitable security protocols, including various encryption algorithms. Examples of such networks are disclosed, for example, in U.S. Pat. No. 10,499,250 (Turner et al.), entitled “RF client for implementing a hyper distribution communications protocol and maintaining a decentralized, distributed database among radio nodes” which is incorporated herein by reference in its entirety.

The above description of the present invention is illustrative, and is not intended to be limiting. It will thus be appreciated that various additions, substitutions and modifications may be made to the above described embodiments without departing from the scope of the present invention. Accordingly, the scope of the present invention should be construed in reference to the appended claims.

What is claimed is:

1. A system for taking an electrocardiogram of a subject, comprising:
a wireless communications system;
a first device which is disposable on a first limb of the subject, said first device containing a first tangible, non-transient memory and a first sensor which measures a first electrophysiological signal of the user;
a second device which is disposable on a second limb of the subject, said second device containing a second sensor which measures a second electrophysiological signal of the user and which is in communication with said first device via said wireless communications system; and
a software program which is resident in said first memory device and which produces an electrocardiogram from the first and second electrophysiological signals measured by the first and second sensors.

2. The system of claim 1, wherein said first device is a smart watch.

3. The system of claim 1, wherein said first device is a smart phone.

4. The system of claim 1, wherein said second device includes an arm band.

5. The system of claim 1, wherein each of the first and second electrophysiological signals is an electrical potential, and wherein said software determines the electrical potential difference between the first and second electrophysiological signals.

6. The system of claim 1, wherein the software program is a software client.

7. The system of claim 1, wherein the software program is installed in the memory of the first device and includes a communications module which establishes and maintains wireless communications with the second device.

8. The system of claim 7, wherein the software program further includes a data processing module which accepts EKG data from the first and second sensors and processes that data, thereby obtaining processed data.

9. The system of claim 8, wherein the software program further includes a graphics module which accepts processed data from the data processing module and which operates on the accepted data to generate graphical data therefrom.

10. The system of claim 9, wherein said graphics module communicates said graphical data to a display on the first device.

11. The system of claim 9, wherein said software program communicates data obtained from the first and second electrophysiological signals to a healthcare professional.

12. The system of claim 9, wherein said software program communicates information derived from the first and second electrophysiological signals to a healthcare professional.

13. The system of claim 1, wherein said second electrophysiological signal is the same as said first electrophysiological signal.

14. The system of claim 1, wherein said second electrophysiological signal is different from said first electrophysiological signal.

15. The system of claim 1, further comprising:
a third device containing a third sensor which measures a third electrophysiological signal of the user and which is in communication with at least one of said first and second devices via said wireless communications system, wherein the third electrophysiological signal is the same as, or different from, the first and second electrophysiological signals.

16. The system of claim 15, wherein said first, second and third devices from a mesh network.

17. A method for monitoring the cardio state of a subject, comprising:
disposing a first device on a first limb of the subject, wherein said first device is a mobile communications device equipped with a first sensor for capturing EKG data from the subject;
disposing a second device on a second limb of the subject, wherein said second device is equipped with a second sensor for capturing EKG data from the subject, and wherein said second device is in wireless communications with said first device;
receiving captured EKG data from the first and second sensors;
operating on the received data to generate at least one graphical object illustrating the cardio state of the subject; and
displaying the graphical object on a display associated with the first device.

18. The method of claim 17, wherein the graphical object is an electrocardiogram.

19. The method of claim 17, further comprising:
periodically updating the graphical object with further instances of received data.

20. A system for taking an electrocardiogram of a subject, comprising:
a host device equipped with a display, a first sensor, tangible, non-transient memory device, and a local database;
a software client installed in said memory device, said software client being equipped with a communications module, a data processing module and a graphical user interface (GUI);
a wireless communications system;
a first device which is disposable on a first limb of the subject, said first device containing a first tangible, non-transient memory and a first sensor which measures a first electrophysiological signal of the user;
a second device which is disposable on a second limb of the subject, said second device containing a second sensor which measures a second electrophysiological signal of the user and which is in communication with said first device via said wireless communications system; and
a software program which is resident in said first memory device and which produces an electrocardiogram from the first and second electrophysiological signals measured by the first and second sensors.
